(19) United States
(12) Patent Application Publication
(10) Pub. No.: US 2006/0166287 A1
(43) Pub. Date: Jul. 27, 2006

(54) Method for Identifying compounds for suppressing metastasizing tumor cells

(76) Inventors: Helmut Ponta, Weingarten (DE); Peter Herrlich, Karlsruhe (DE); Chen Linfeng, Boston, MA (US); Veronique Orian-Rousseau, Riettershoffen (FR)

Correspondence Address: FRIEDRICH KUEFFNER, 317 MADISON AVENUE, SUITE 910, NEW YORK, NY, 10017, US

(21) Appl. No.: 10/516,231

(22) PCT Filed: May 30, 2003
(86) PCT No.: PCT/EP03/05665

§ 371 Date: Aug. 1, 2005

(30) Foreign Application Priority Data
May 29, 2002 (DE) 102 23 696.8

(51) Int. Cl. G01N 33/574; C07K 16/30

(52) U.S. Cl. 435/7.23; 530/388.8

(57) ABSTRACT

The present invention relates to a method for identifying compounds which suppress and/or delay the development of invasive or metastasizing tumor cells of the colon, of the pancreas, of the ovaries or of the thyroid.

The present invention relates to a method for identifying compounds which suppress and/or delay the development of invasive or metastasizing tumor cells of the colon, of the pancreas, of the ovaries or of the thyroid.

An important property of cells of the multicellular organism is their ability to cease proliferating when the space assigned to them is filled. This process, which is referred to as contact inhibition, has been known for a long time. The signal to which the cells respond during the contact inhibition process might be contact of the cells with one another or contact between the cells and the extracellular matrix system. The contact inhibition process requires the presence of one or more surface sensors which communicate with the cell nucleus, bringing the cell cycle to a stop in the G1 phase and, in this way, bring about inhibition of growth. In vitro, in cell culture, normal cells multiply until they out the surface of the culture vessel, then remain stationary in the G1/G0 phase when a dense single-cell layer has formed (contact inhibition). A further manifestation of the cessation of growth is their inability to form colonies when they are embedded in a matrix of sufficient density (soft agar). The complete mechanisms by which the cell ceases to grow are still substantially unknown. The loss of contact inhibition and formation of colonies in soft agar are properties characteristic of cancer cells.

The receptor thyrosine kinase c-Met and its ligand HGF/SF control various cellular processes which are essential for survival. Destruction of the genes of the receptor thyrosine kinase in mice leads to death of embryos owing to lack of development of the placenta (HGF/SF) and a reduced development of various epithelial organs. Analyses of these genetically modified mice and studies with organ and cell cultures have revealed that the receptor thyrosine kinase involved in an invasive growth and cellular migration, in proliferation and differentiation, in mesenchymal-epithelial communication and tubular endothelial organization and morphogenesis. The receptor thyrosine kinase c-Met is expressed in particular in epithelial cells, whereas its ligand HGF/SF is secreted in mesenchymal cells. The protein c-Met has two essential tyrosine residues, Y1349 and Y1356, which serve through phosphorylation as so-called docking sites for various signal components. In animal systems, c-Met catalyzes invasive and metastatic properties. Amplification of the c-Met gene and overexpression of c-Met have been observed in human cancer cells from the colon, the pancreas, the ovaries and the thyroid. Hereditary renal carcinomas, just like carcinomas in the head and neck, have been associated with the c-Met gene locus.

However, invasive and methasthatic growth of tumors is not only influenced by the HGF/SF/MET system and its intracellular transduction pathways; on the contrary, other growth factors, the extracellular matrix, adhesion molecules and their intracellular complexes are also determined as responsible for the growth of tumors. Numerous studies have been carried out to identify and select relevant components.

It was possible through the differential expression of epitopes in metastatic and non-metastatic cancer cells of the pancreas to identify a metastasis-associated isoform of CD44. The name CD44 represents a family of class I transmembrane proteins produced by very pronounced alternative splicing of the exons v1 to v10.

A causal connection between CD44 isoforms and the development of metastatic tumor cells was documented by transferring CD44 isoforms into various non-metastatic cell lines. Isoforms which have the sequences encoded by exons v4 to v7 or only v6 and v7 were sufficient for development of a metastatic potential. Antibodies directed against a v6-encoded epitope or CD44v6 antisense RNA suppressed tumor growth in vivo and metastatic dissemination of cancer cells.

Elucidation of the molecular interplay of the individual factors in the development of invasive or metastasizing tumor cells, preferably of the colon, of the pancreas, of the ovaries or of the thyroid, are therefore of high medical relevance. Likewise, the identification of compounds able to prevent and/or delay the development of invasive or metastasizing tumor cells is of enormous relevance.

The present invention relates to a method for identifying compounds which suppress and/or delay the development of invasive or metastasizing tumor cells of the colon, of the pancreas, of the ovaries or of the thyroid, where the formation of a trimeric complex comprising a receptor thyrosine kinase c-Met encoded by the c-Met gene, an isoform of the cell surface protein CD44 comprising the exon sequence v6 and the c-Met ligand HGF/SF is prevented and/or delayed through the interaction of the putative compounds with at least one of the three components mentioned.

The possibilities in this connection are extracellular addition and/or intracellular provision of the putative compounds in the method of the invention.

It has been possible to show that the presence of a CD44 isoform comprising a v6 sequence is absolutely necessary for c-Met activation and signal transmission. The receptor thyrosine kinase c-Met, the ligand HGF and a CD44 variant comprising a v6 sequence form a trimeric complex. This complex formation is suppressed by antibodies which specifically recognize the v6 epitopes.

One variant of the method of the invention is distinguished by
a) provision of a cell culture of a tumor cell line of cells of the colon, of the pancreas, of the ovaries or of the thyroid, where this tumor cell line culture is capable of expression of the receptor thyrosine kinase c-Met, of an isoform of the cell surface protein CD44 comprising the exon sequence v6 and of the c-Met ligand HGF/SF,
b) extracellular provision of this cell culture with a compound which interacts specifically with at least one component of the trimeric complex,
c) provision of the cell culture treated in this way with an appropriate culture medium for growth of the cells, and
d) identification of a compound from b) as one which prevents and/or suppresses the formation of the trimeric complex, so that the cells of the tumor cell line no longer grow.

In an advantageous embodiment of the method of the invention there is intracellular provision in step b) of a compound, where this compound is introduced into the cells actively by membrane-transport proteins or passively by diffusion, or is synthesized directly by the cells themselves.

In a preferred variant of the method of the invention, a peptide is used as compound. In a particularly preferred embodiment, a specific antibody against the exon sequence v6 of the CD44 isomer is used.

The present invention relates to the compounds which are identified by means of the method of the invention of the type explained above. In this connection, the compounds of the invention are distinguished by specifically binding to the exon sequence v6 of the cell surface protein CD44. The compounds of the invention are preferably peptides.

The present invention further relates to the use of the compounds of the invention for the treatment of cancers. The invention likewise encompasses a use of the compounds to produce compositions for the treatment of cancers. The present invention is explained in detail by the following examples, which do not, however, have a limiting effect.

C-Met Activation

Two metastatic cell lines, BSp73ASML and HT29, which express CD44 proteins with a v6 sequence and c-Met were employed. Said cell lines were starved and then treated with HGF in the presence or absence of a CD44v6-specific antibody and harvested 5 minutes later. C-Met was then immunoprecipitated, fractionated by SDS-PAGE electrophoresis and employed in a Western blot analysis using phosphotyrosine-specific or c-Met-specific antibodies (FIG. A1). Whereas HGF led to increased c-Met tyrosine phosphorylation, the CD44v6-specific antibody completely prevented this activation. Antibodies which recognize the N-terminal domain of CD44 (IM7 and J173) did not show this effect. Antibodies which recognize the smallest unit of CD44 (CD44s) or a CD44 variant comprising the v6 sequence but not the exon 15 sequence (epitope 5G8) are also ineffective. On this basis, CD44v6 is absolutely necessary for c-Met activation.

Comparable results are shown in FIG. 1B and 1C. HGF-dependent metastatic properties of tumor cells were suppressed in the presence of anti-v6 antibodies (FIG. 1B and C). The anti-v3 antibody BBAll did not suppress metastasis, so that the exon v3 is presumably not involved in this process.

CD44 isoforms comprising exon v6 are necessary and sufficient for cooperation with c-Met.

Cooperation with CD44 and c-Met

In order to investigate which CD44 variants are necessary for cooperation with c-Met, an exon-specific RT-PCR was carried out in two different tumor cell lines (FIG. 2). The HT29 cell line expresses in particular 4 isoforms of CD44, namely CD44v4-10, CD44v2, 3, 8-10, CD44v2, 9, 10 and CD44s. The strongest signal (in FIG. 2: lane C) was given by the isoforms CD44v4-10. The dominant isoforms of CD44 in the BSp73ASML cell line are CD44v4-7, CD44v6, 7, and CD44s.

To determine which of the CD44 isoforms interact with c-Met, c-DNA expression clones which encode individual CD44 isoforms were transfected into the tumor cell line BSp73AS. The BSp73AS cell line expresses exclusively CD44s (FIG. 2) and comparable contents of c-Met compared to the metastatic cell line BSp73ASML (FIG. 3C). The transfected recipient cell activates a c-Met signal transmission measured by the phosphorylation of ERK (FIG. 3B, lane 1). Additional overexpression of CD44s does not enhance this signal response. (FIG. 3B, lane 2). The transfection experiments clearly show that HGF/SF-dependent c-Met phosphorylation and ERK activation (FIG. 3B) was achieved by all CD44 variants which comprised an exon v6 sequence, or a protein which comprised only the v6 region (CD44v6). The structure of the CD44 variants which were introduced into the cell lines is shown in FIG. 3A. Examples of the expression of CD44v4-7 transfectants are depicted in FIG. 2. This analysis also comprises exon v6-containing cDNA clones lacking the exon 15 sequence (epitope 5G8; Δ15) and leading to a metastatic behavior of the BSp73AS cells. The presence or absence of exon 15 has no influence on the cooperation of CD44 with c-Met.

It was thus possible to show that the presence of the v6 sequence of a CD44 isoform is sufficient and obligatory for c-Met signal transmission.

Investigations with cDNA clones which encode CD44v1-10 isoforms but have a deletion of v6 have shown that these cells do not bring about any c-Met-mediated ERK activation (FIG. 3B).

Heparan Sulfate Modification of CD44

A heparan sulfate modification of CD44 is unnecessary for activation of c-Met by HGF/SF, as shown in FIG. 4.

Activation of Pro-HGF to the Active Form of HGF

HGF/SF is secreted as single-stranded precursor which is processed by serum proteinases (Shimomura, 1995) or plasminogen activators of the urokinase type (Naldini, 1995) into an active a form and β subunits. Investigations into whether CD44 is involved in the activation of HGF was employed HGF precursor which was purchased and isolated from Drosophila Schneider cells (S2) for the activation of c-Met (FIG. 5). The pro form of HGF is, however, despite the presence of competent CD44 isoforms, unable to activate c-Met.

CD44v6-Isoforms, c-Met and HGF/SF Associate in a Trimeric Complex

To demonstrate a close association of said 3 components in a cellular membrane, coimmunoprecipitation studies were carried out. The coprecipitation was carried out without previous crosslinking and after stabilization by crosslinking with dithiobis(sulfosuccinimidyl propionate); DTSSP. In addition, pairwise precipitation and detection in Western blots, and immunoprecipitation in a non-denaturing gel and detection of the components by Western blotting took place. The results are depicted in FIG. 6A and show the existence of an induced trimeric complex. It was possible to show by 2-dimensional resolution under non-reducing conditions in the first dimension that the crosslinked complex comprises all 3 components (FIG. 6A).

It was possible to prevent the induced formation of the immunoprecipitated complex by previous addition of an antibody directed against the exon v6 epitope of CD44; but not by antibodies which are specific for the N-terminal region of CD44 (FIG. 6B).

### KEY TO THE FIGURES

FIG. 1:

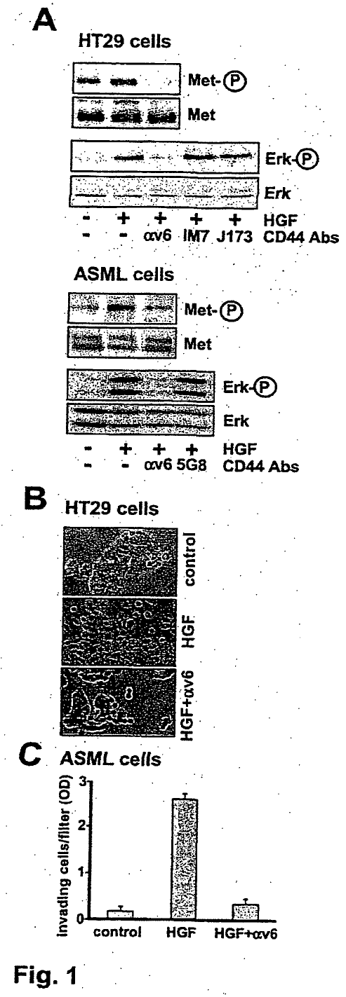

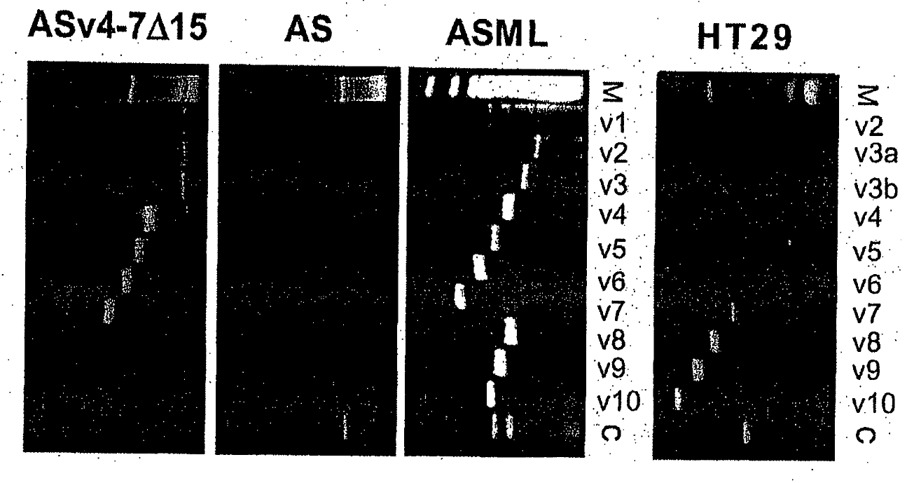

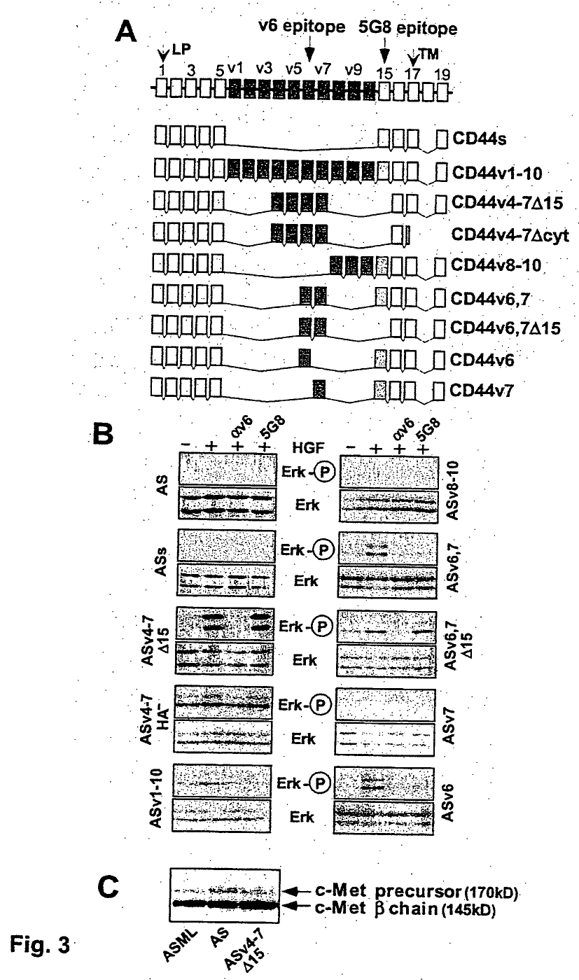

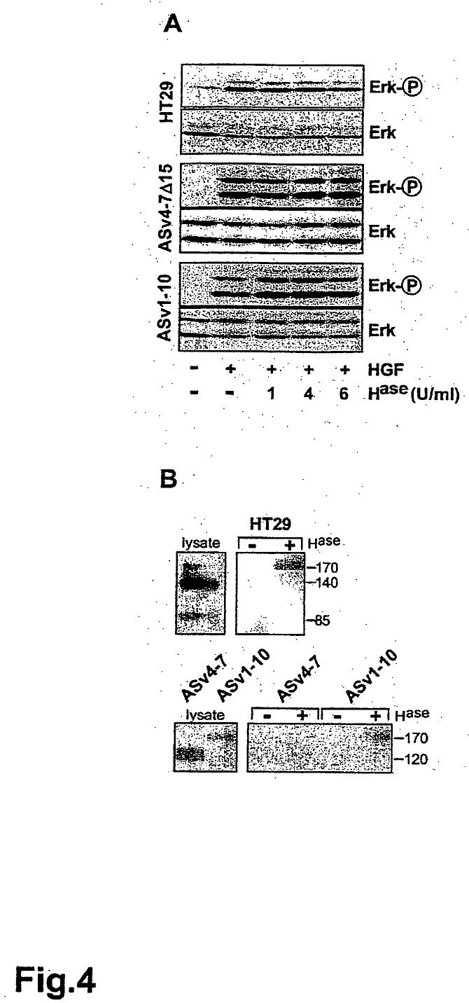

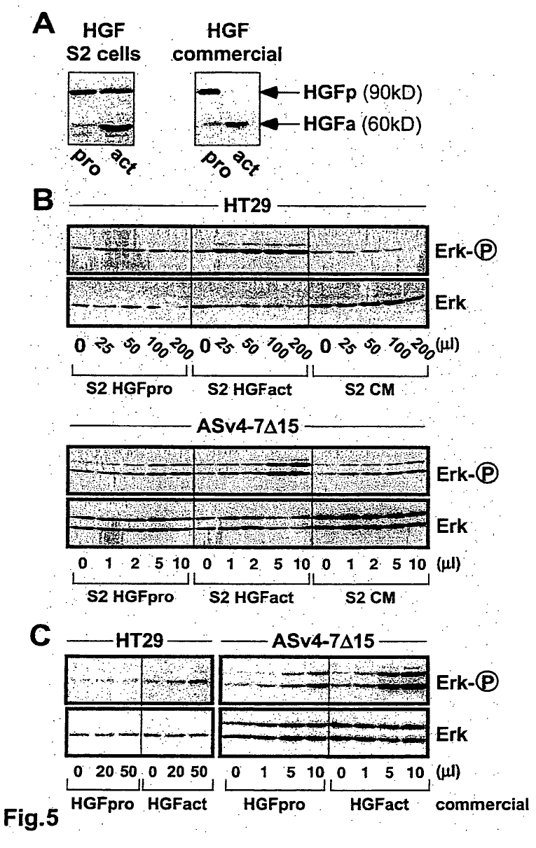

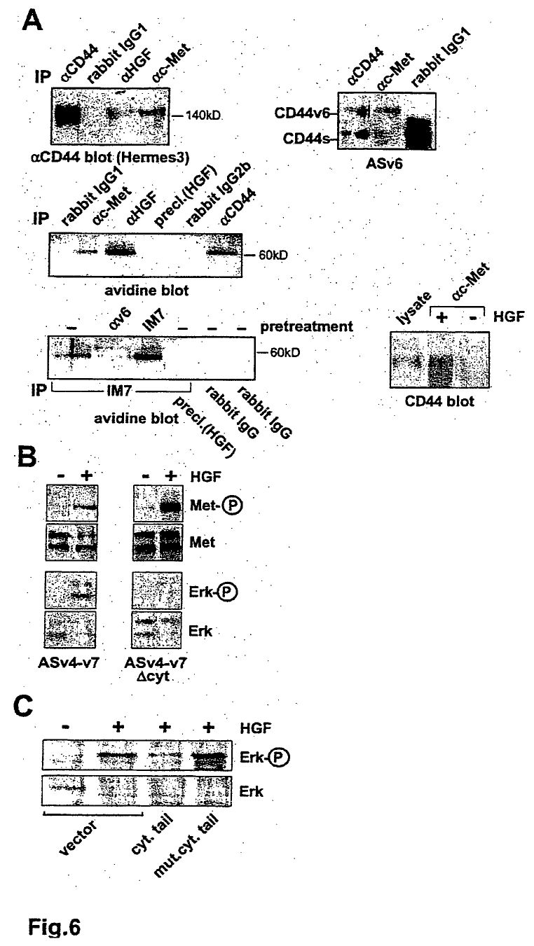

(A) Western blot analysis of the HT29 and BSp73ASML tumor cell lines without and with phosphotyrosine-specific or c-MET-specific antibodies.

(B) Micrographs of the HT29 tumor cell line under normal conditions after treatment with HGF and after pretreatment with a v6-specific antibody and subsequent treatment with HGF. Cells treated with the ligand HGF have separated from one another, are scattered and have migrated. Pretreatment with the v6-specific antibody has prevented cell separation and migration. The cells remain closely associated together.

(C) Diagrammatic representation of the chemotaxis of metastasizing cells of the BSp73AASML cell line with addition of HGF in a Matrigel. 50 ng/ml HGF were placed on the base of a Transwell reaction chamber. The ASML cells were put in the upper reaction chamber which is separated by a filter coated with Matrigel. Without HGF addition, the cells do not migrate; however, migration of the cells is clearly evident through addition of HGF. This cell migration is completely inhibited by addition of the CD44 antibody.

FIG. 2:

Electrophoretic fractionation of RT-PCR analyses of the HT29, BSp73ASML, BSp73AS and BSp73Asv4-7Δ15 tumor cell lines. The various CD44 isoforms are evident in the individual cell lines. The lane identified by M shows a molecular weight standard, lanes v2 to v10 represent the CD44 isoforms with the various exon sequences. To estimate the intensity and size of the individual RT-PCR products, an RT-PCR product with primers from a constant region of CD44 was loaded in lane C.

FIG. 3:

(A) Diagrammatic representation of the various CD44-isoforms comprising corresponding exon sequences, which are identified by v.

(B) Western blot analysis of the BSp73AS cell line, and transfected cells of this cell line which express different CD44 isoforms. The activation of c-Met is represented by the phosphorylation of ERK. Addition of HGF to the individual cells is indicated by − and +. Likewise, specific antibodies against the v6 sequence (αV6) or against the exon 15 epitope (5G8) were added to the HGF-treated cells.

(C) Western blot analysis to visualize c-Met (precursor and P chain) in the BSp73ASML, BSp73AS and BSp73Asv4-7Δ15 cell lines.

FIG. 4:

(A) Depiction of ERK phosphorylation with and without addition of HGF in a Western blot analysis of the HT29, BSp73Asv4-7Δ15 and BSp73Asv1-10 cell lines, the cells having been additionally treated without or with heparinase (1, 4 or 6 U/ml) in order to investigate the influence of a heparan sulfate modification. A heparan sulfate modification of CD44 has no influence on the activation of c-Met by HGF.

(B) Western blot analysis of the HT29, BSp73Asv4-7 and BSp73Asv1-10 cell lines without and with heparinase treatment with a CD44-specific antibody which makes it possible to recognize a heparan sulfate modification of CD44 through binding to heparan residues resulting from heparinase digestion. The CD44 isoforms of said cell lines were not subject to heparan sulfate modification. A lysate of CD44v1-10-transfected BSp73AS cells served as positive control. The main Cd44 isoform could not be precipitated in lysates of HT29 cells. HT29 cells harbor modified and unmodified CD44 proteins.

FIG. 5:

(A) Western blot to visualize the cleavage of the HGF precursor HGFp, enriched from Drosophila Schneider cells (S2), and commercially purchased HGFp, into the active form HGFa through addition of serum proteinases.

(B) Western blot analyses to investigate the cleavage of the precursors pro-HGF (HGFp; enriched from Drosophila Schneider cells (S2)) into the active form HGFa by means of CD44 in the HT29 and BSp73Asv4-7Δ15 cell lines. The pro form is unable, despite the presence of competent CD44 isoforms, to mediate ERK phosphorylation by c-Met activation. In contrast thereto, processed, active HGF is capable, with the assistance of competent CD44 isoforms, of c-Met activation. This shows that a CD44-c-Met cooperation does not take place via cleavage of the HGF pro form into the active HGF form.

(C) Western blot analysis to investigate the cleavage of the precursor pro-HGF (HGFp; commercial) into the active form HGFa by means of CD44 in the HT29 and BSp73Asv4-7Δ15 cell lines. The pro form is unable, despite the presence of competent CD44-isoforms, to mediate ERK phosphorylation by c-Met activation. In contrast thereto, processed, active HGF is capable, with the assistance of competent CD44 isoforms, of c-Met activation. This shows that CD44-c-Met cooperation does not take place via cleavage of the HGF pro form into the active HGF form.

FIG. 6:

(A) To investigate the association of a trimeric complex of CD44v6 isoforms, c-Met and HGF/SF, coimmunoprecipitation experiments were carried out. The coprecipitation was carried out without and with crosslinking (after stabilization with dithiobis(sulfosuccinimidyl propionate); DTSSP). Pairwise precipitation, and immunoprecipitation in a non-denaturing gel and detection in Western blots took place. CD44s could not be precipitated in lysates of the BSp73AS cell line. It was possible for a trimeric complex to be formed in lysates of transfected cells which additionally comprised CD44v6 isoforms. In the absence of HGF, coprecipitation of c-Met and CD44 was not possible. From lysates of the HT29 cell line it was possible to precipitate a single CD44 band with a size of 140 kDa together with the trimeric partner. The 140 kDa protein corresponds to the main isoform of CD44 in HT29 cells. It was possible to detect all three components of the complex by two-dimensional resolution under non-reducing conditions in the first fractionation.

(B) Western blot analysis which demonstrates that the addition of antibodies specifically against the exon v6 epitope of CD44 prevents formation of the immunoprecipitated trimeric complex. Antibodies against the N-terminal domain of CD44 do not show this effect.

1. A method for identifying compounds which suppress and/or delay the development of invasive or metastasizing tumor cells of the colon, of the pancreas, of the ovaries or of the thyroid, where the formation of a trimeric complex comprising a receptor thyrosine kinase c-Met encoded by the c-Met gene, an isoform of the cell surface protein CD44 comprising the exon sequence v6 and the c-Met ligand HGF/SF is prevented and/or delayed through the interaction of the putative compounds with at least one of the three components mentioned.

2. The method as claimed in claim 1, comprising extracellular addition and/or intracellular provision of the compounds.

3. The method as claimed in claim 1, comprising
e) provision of a cell culture of a tumor cell line of cells of the colon, of the pancreas, of the ovaries or of the thyroid, where this tumor cell line culture is capable of expression of the receptor thyrosine kinase c-Met, of an isoform of the cell surface protein CD44 comprising the exon sequence v6 and of the c-Met ligand HGF/SF,
f) extracellular provision of this cell culture 30 with a compound which interacts specifically with at least one component of the trimeric complex,
g) provision of the cell culture treated in this way with an appropriate culture medium for growth of the cells, and
h) identification of a compound from b) as one which prevents and/or suppresses the formation of the trimeric complex, so that the cells of the tumor cell line no longer grow.

4. The method as claimed in claim 1, comprising intracellular provision in step b) of a compound, where this compound is introduced into the cells actively by membrane-transport proteins or passively by diffusion, or is synthesized directly by the cells themselves.

5. The method as claimed in claim 1, wherein a peptide is used as compound.

6. The method as claimed in claim 1, wherein a specific antibody against the exon sequence v6 of the CD44 isomer is used.

7. A compound obtained by a method as claimed in claim 1.

8. The compound as claimed in claim 7, wherein it specifically binds to the exon sequence v6 of the cell surface protein CD44.

9. The compound as claimed in claim 4, wherein it is a peptide.

10. (canceled)

11. (canceled)

12. A compound obtained by a method as claimed in claim 2.

13. A compound obtained by a method as claimed in claim 3.

14. A compound obtained by a method as claimed in claim 4.

15. A compound obtained by a method as claimed in claim 5.

16. A compound obtained by a method as claimed in claim 6.

17. The compound as claimed in claim 12, wherein it specifically binds to the exon sequence v6 of the cell surface protein CD44.

18. The compound as claimed in claim 13, wherein it specifically binds to the exon sequence v6 of the cell surface protein CD44.

19. The compound as claimed in claim 14, wherein it specifically binds to the exon sequence v6 of the cell surface protein CD44.

20. The compound as claimed in claim 15, wherein it specifically binds to the exon sequence v6 of the cell surface protein CD44.

21. The compound as claimed in claim 16, wherein it specifically binds to the exon sequence v6 of the cell surface protein CD44.
